(19) United States
(12) Patent Application Publication
(10) Pub. No.: US 2020/0400638 A1
(43) Pub. Date: Dec. 24, 2020

(54) METHOD FOR THE QUANTIFICATION OF THE TOTAL GLUTEN CONTENT OF GRAINS IN FOOD SAMPLES

(71) Applicant: R-Biopharm AG, Darmstadt (DE)

(72) Inventors: Thomas Weiß, Darmstadt (DE); Markus LACORN, Oestrich-Winkel (DE); Thomas HEKTOR, Griesheim (DE)

(73) Assignee: R-Biopharm AG, Darmstadt (DE)

(21) Appl. No.: 16/968,473

(22) PCT Filed: Jan. 3, 2019
(86) PCT No.: PCT/EP2019/025002

§ 371 Date: Aug. 7, 2020

(30) Foreign Application Priority Data
Feb. 8, 2018 (EP) 18000114.1

(51) Int. Cl. G01N 33/02

(52) U.S. Cl. CPC G01N 33/02; G01N 2333/415

(57) ABSTRACT

The invention relates to a method for the quantification of the total gluten content of grains in food samples. Areas of application are primarily the food industry, service laboratories and government test laboratories or biotechnology. The invention aims to quickly and cost-effectively determine the total gluten content in foods with just one measurement. We include a method with which, based on the detection of prolamins and glutelins, all potentially coeliac-relevant gluten protein fractions in food samples are quantified as a total corresponding to their mass. The method is based on the joint use of specific prolamin and glutelin antibodies as a combined conjugate according to the invention for the detection of the total gluten content. In addition, the individual antibody conjugates are thinned so that the contribution of the individual antibodies to the total signal strength corresponds to the proportion of the gluten fraction that they each detect.

### CROSS-REFERENCE TO RELATED APPLICATIONS

This application is the United States National Stage under 35 U.S.C. 371 of PCT International Patent Application No. PCT/EP2019/025002, filed on Jan. 3, 2019, and claiming the benefit of priority of European Patent Application No. 18000114.1, filed on Feb. 8, 2019.

### BACKGROUND OF THE INVENTION

### Field of the Invention

The invention relates to a method for quantifying total gluten from cereals in food samples. Areas of application are, above all, the food industry, service laboratories and governmental control laboratories or biotechnology.

### Description of Related Art

With an annual harvest of 2.5 billion tonnes, cereals are the most important foodstuff of humans. The range of foods made from grain, supplemented with grain products or treated with grain ingredients is vast. Some grains, such as wheat, barley and rye, contain large amounts of gluten, which is beneficial for baking but causes intolerance in certain people, known as celiac disease. In addition, another intolerance has been observed in the form of “Non Celiac Gluten Sensitivity”, but this has not yet been fully researched.

The prevalence of coeliac disease varies according to culture; the worldwide average is 1:270, which is about 250 million people. Celiac disease is a partially autoimmune disease that causes damage to the small intestine, which manifests itself in diarrhoea, vomiting, malnutrition and weight loss. Since there is no cure for the disease, the only option for the sufferer is a strict gluten-free diet. Celiac disease patients who follow such a diet live largely symptom-free. The patients are therefore dependent on food that contains no or very little gluten.

The Codex Alimentarius, established in 1963 by the World Health Organization (WHO) and Food and Agricultural Organization of the United Nations (FAO), describes the requirements for foods that are considered “gluten-free”. The currently valid standards define foods as “gluten-free” if they contain less than 20 mg gluten per kg of food (Codex Standard 118-1979). Other countries and regions, such as the EU, USA and Canada have adapted their legal regulations to this codex limit. Food manufacturers who wish to ensure the supply of “gluten-free” products to coeliac patients must check and comply with this limit value for their products.

Gluten is a heterogeneous protein mixture of alcohol-soluble prolamines and alcohol-insoluble glutelines. Gluten proteins are found in all Triticum species (e.g. wheat, spelt, emmer, einkorn), rye and barley as well as in their crossbreeds and are differentiated according to the type of grain. For example, in wheat the prolamines are called gliadins and the glutelins are divided into low molecular weight glutenin subunit (LMW) proteins and high molecular weight glutenin subunit (HMW) proteins. Historically, only the prolamines have been considered “toxic” to celiac disease patients. Recently, however, it has been found that LMW and HMW proteins are also toxic (Tye-Din et al. 2010).

The most widely used detection method for the detection of gluten proteins is currently the enzyme-linked immunosorbent assay (ELISA). Extraction solutions containing reducing substances (e.g. β-mercaptoethanol, sulfite, thiosulfite), denaturing substances (e.g. guanidine hydrochloride, urea) and/or detergents (e.g. Tween, Triton-X100, sodium dodecyl sulfate) are used for the extraction of food samples. One of these extraction methods is the so-called cocktail extraction solution (EP 1 424 345 A1; licence with R-Biopharm AG). All extraction solutions are used together with ethanol or isopropanol.

Commercially available immunological detection systems are mostly based on monoclonal antibodies (e.g. Skerritt, R5, G12 and Alpha20). Most of these antibodies mainly or exclusively detect the better soluble prolamines (Wieser et al. 2014).

As an example, one of the most frequently used measurement systems for the quantification of gluten, the so-called Mendez System consisting of Cocktail Extraction and R5 ELISA, is explained in more detail below. This system is commercially available as Cocktail (patented EP 1 424 345 A1) R7006 (R-Biopharm AG, Darmstadt, Germany) and RIDASCREEN® Gliadin R7001 (R-Biopharm AG). The homogenized food sample is extracted at 50° C. using Cocktail (patented) and then ethanol is added. After dilution of the obtained extract, the prepared sample is incubated on a microtiter plate coated with monoclonal antibody R5. After washing, an R5-horseradish peroxidase conjugate is incubated. After a further washing step, the colour reaction takes place by adding a substrate/chromogen solution. The reaction is stopped by sulfuric acid and the optical density (OD) is measured by a photometer. The quantification is performed using calibrators with known gliadin content. From the gliadin content thus obtained, the total gluten content is calculated by multiplication with the factor 2.

This multiplication is prescribed by the Codex Alimentarius (CODEX-STAN 118—1979). However, it is becoming increasingly clear that a general factor 2 leads to miscalculation in many cases (Wieser and Köhler, 2009). Wheat flours, for example, can differ significantly both in their total protein content and in the composition of the gluten proteins (Hajas et al., 2017). If only the prolamin content is determined, this can lead to falsified results when calculating the total gluten content if the composition of the individual gluten protein fractions differs.

Furthermore, depending on how the grain is processed to the “gluten-free” product, certain gluten fractions (prolamines and glutelines) can be selectively depleted or enriched, so that if the gluten fractions are only partially determined, significant false results may be obtained (Wieser and Köhler 2009).

A joint detection of prolamines and glutelines that could solve these problems is not described in prior art. For example, EP 1 1779 115 B1 reveals antibodies against T-cell recognition sequences in gliadin (prolamine in wheat) or in LMW and HMW (gluteline in wheat) and their use in ELISA assays. However, there is no joint detection, only either prolamine or LMW or HMW detection. The separate detection is also emphasized as being particularly advantageous. Therefore there is no sufficient quantification of total gluten from wheat and/or rye and/or barley in an ELISA.

In addition, Skerritt et al. (1989) published several antibody combinations that can partially detect prolamins, LMW and HMW. However, in each case only one monoclonal antibody was coated and one monoclonal antibody was used in the conjugate; thus, no combination of several antibodies in one cavity or in one conjugate took place. Correspondingly, none of the tested systems showed a sufficiently uniform detection of the different gluten fractions and thus also no sufficient quantification of total gluten. In fact, it is probably only a question of cross reactivities of the antibodies against the other gluten fractions, as it was not intended to produce specific antibodies against the individual fractions. The commercial ELISA ALLER-TEK™ Gluten ELISA Assay was developed from one of the antibody combinations described by Skerritt et al (1989) (Anonymus, 2014). The above described limitations of the ELISA systems described by Skerritt et al. therefore also apply to this commercial system.

In fact, this was confirmed by Lexhaller et al. (2016) for the commercial ELISA of ALL-TEK™ Gluten ELISA assay and for four other commercial sandwich ELISA systems, so that all five commercial ELISA systems cannot satisfactorily determine total gluten.

Few commercial ELISA assays use polyclonal antibodies (Wieser et al. 2014). Even when immunized with total gluten, antibody reactivity is randomly generated. This makes it impossible to adjust the system afterwards, so that each gluten protein fraction can be quantified according to its mass fraction.

Due to the disadvantages mentioned above, these methods are no longer the means of choice for determining the actual and accurate total gluten protein content in food.

### BRIEF SUMMARY OF INVENTION

The aim of the invention is to determine the total gluten content in food quickly and inexpensively with only one measurement, whereby each gluten protein fraction is to be quantified according to its mass fraction.

From this, the task of the present invention is derived to develop a method with which, based on the detection of prolamines and glutelines, all potentially celiac-relevant gluten protein fractions in food samples are quantified as a sum according to their masses, whereby the quantification is carried out by combining several antibodies in a cavity and in a conjugate. The method is to be used for the determination of the total gluten content of wheat samples. Related gluten fractions in other Triticum species, rye and barley should also be quantifiable with the method.

The present method is based on the joint use of specific prolamin and glutelin antibodies as a combination conjugate for the detection of total gluten in accordance with the invention. To quantify the total gluten content, a combination of specific binding reagents against prolamine and gluteline is used in a single analytical run, whereby these specific binding reagents are mixed together in one unit. The individual antibody conjugates are diluted in such a way that the contribution of the individual antibodies to the total signal strength corresponds to the proportion of the gluten fraction they each detect.

### BRIEF DESCRIPTIONS OF THE DRAWINGS

FIG. 1: Schematic function of a lateral flow assay in sandwich format.

FIG. 2: Test the reactivity of the combination conjugate against the different wheat flour fractions.

FIG. 3: Test the reactivity of the combination conjugate against the different wheat flour fractions. Measured concentrations for different wheat flour fractions at a target value of 20 ng/ml.

FIG. 4: Test of the reactivity of the combination conjugate against various commercial wheat flours and some pure wheat varieties, each with a gluten protein content determined by HPLC.

FIG. 5: Test of the reactivity of the combination conjugate against various commercial wheat flours and pure wheat varieties, whose gluten protein content was determined by HPLC. Measured concentrations at a target value of 20 ng/ml.

FIG. 6: Test of the reactivity of the combination conjugate against various commercial rye flours and pure rye varieties, whose gluten protein content was determined by HPLC, and purified rye fractions.

FIG. 7: Test of the reactivity of the combination conjugate against various commercial rye flours and pure rye varieties, whose gluten protein content was determined by HPLC, and purified rye fractions. Measured concentrations at a target value of 20 ng/ml.

FIG. 8: Test of the reactivity of the combination conjugate against various commercial barley flours and pure barley varieties, the gluten protein content of which was determined by HPLC, and purified barley fractions.

FIG. 9: Test of the reactivity of the combination conjugate against various commercial barley flours and pure barley varieties, whose gluten protein content was determined by HPLC, and purified barley fractions. Measured concentrations at a target value of 20 ng/ml.

### DETAILED DESCRIPTION OF THE INVENTION

Prior to the total gluten determination, the gluten proteins are extracted from the food. For this purpose, an extraction method is used which, under reductive conditions with the addition of chaotropic salts in the presence of aliphatic short-chain alcohols, releases gluten proteins from the food matrix.

The resulting extract is then used for the total gluten determination using specific binding reagents such as antibodies, antibody fragments, nanobodies, aptamers, specific receptors, T-cell receptors and/or Molecularly Imprinted Polymers (MIP). Preferably, the total gluten is determined in an antibody-based detection method, e.g. in an ELISA (sandwich or competitive or direct format) or lateral flow format or flow through.

For a better understanding, the general principle of a sandwich ELISA is explained below. In a sandwich ELISA, the antigen (analyte) is specifically bound in a sandwich fashion by two antibodies. The first antibody (capture antibody) is bound to the solid phase of the wells (cavities) of a microtiter plate. The sample with the antigen to be detected is then added to the wells and incubated. During this time the antibody bound to the plate binds to the antigen present in the sample. After the incubation phase the plate is washed. The unbound components of the sample are thereby removed. Only the antigen bound to the capture antibody remains. In the next step, a second antibody for detection (detection antibody) is added, which binds to a different site in the antigen than the capture antibody and to the end of which a reporter enzyme is bound. This second antibody therefore also binds to the antigen, only at a different position, and an antibody-antigen-antibody complex, also known as a sandwich complex, is formed. Washing the plate again removes excess, unbound detection antibody. The antigen can then be detected and quantified: A substrate (also called “chromogen”) matching the reporter enzyme is added, which is converted by the reporter enzyme into a reaction product, thus enabling its detection by colour change. Other possibilities instead of the reporter enzyme would be direct fluorescence or chemiluminescence labelling of the detector antibody. For a quantitative determination, a series of known antigen concentrations (standard series) is usually carried along to obtain a calibration curve for the measured signal.

In the case of the present invention, in order to perform a sandwich ELISA, a microtiter plate is coated with a combination of antibodies to wheat prolamine, LMW and HMW proteins prior to the performance of the assay, each well being coated with the mixture of antibodies. The extracted sample is then added. The extracted gluten proteins bind specifically to these capture antibodies. After a washing step, the combined addition of detection antibodies against wheat prolamine, LMW and HMW proteins is carried out according to the invention, whereby the conjugated antibodies are present in a solution (combination conjugate). These detection antibodies are conjugated in such a way that their binding becomes quantifiable. This can be done, for example, by conjugation with a dye-forming reporter enzyme. Furthermore, the conjugated detection antibodies used against prolamines and glutelines in the combination conjugate are mixed according to the invention in such a way that all gluten fractions are measured at the same mass fraction with comparable signal strength.

The quantification is performed by comparing the total signal strength of a sample with the signal strengths of gluten calibrators of known content. An extract of wheat flour, for example, can be used as calibrator material.

The invention is explained in more detail below with concrete embodiment examples, which, however, should not be restrictive for the scope of protection of the invention. Modifications and amendments of the invention which are obvious to a person skilled in the art are accordingly covered by the scope of protection of the patent claims.

As already mentioned in one of the above sections, the total gluten determination is preceded by the extraction of the gluten proteins from the sample to be examined under addition of chaotropic salts in the presence of aliphatic short-chain alcohols. The following is an example of the extraction of wheat gluten proteins from food samples of various kinds with a cocktail extraction solution [0.76 g/L NaCl, 0.224 g/L KCl, 1.42 g/L Na₂HPO₄*2H₂O, 0.272 g/L H₂KO₄P, 191 g/L Guanidine HCl, 18.2 ml/L β-Mercaptoethanol]. (EP 1 424 345 A1) is described in brief.
homogenise a sufficient quantity of the sample (at least 5 g or 5 ml) well (crush carefully, grind finely and mix well or mix the solution well).
for liquid samples: add 2.5 ml cocktail extraction solution (EP 1 424 345 A1) to 0.25 ml of the homogenised sample, close the vessel and mix well
other samples (e.g. soya and quinoa containing food samples): weigh 0.25 g of the homogenised sample and add 2.5 ml cocktail extraction solution (EP 1 424 345 A1), close the vessel and mix well
food samples containing tannin and polyphenols: (e.g. chocolate, coffee, cocoa, chestnut flour, buckwheat, millet and spices): weigh 0.25 g of the homogenised sample, add 0.25 g skimmed milk powder and 2.5 ml cocktail extraction solution (EP 1 424 345 A1), close the vessel and mix well
meat and sausage samples: the gluten distribution in these foods can be very unequal, so weigh 50 g of the sample and homogenise it: weigh 0.25 g of the homogenised sample and add 2.5 ml cocktail extraction solution, close the vessel and mix well
oat samples: the gluten distribution can be very unequal, in addition these samples are difficult to homogenize. Therefore homogenize 200 g sample, the sample preparation should then be carried out at least with the fourfold approach: Weigh 1 g of the homogenized sample and add 10 ml cocktail extraction solution, close the vessel and mix well.

All samples are further treated as described below:
incubate 40 min at 50° C.
allow sample to cool and then add 7.5 ml 80% ethanol (for oat samples: 30 ml 80% ethanol)
close the vessel and shake it upside down for 1 h at room temperature (20-25° C.) or rotate it using a rotator
centrifugation: 10 min, min. 2500 g, at room temperature (20-25° C.) and/or filtration (alternatively transfer 2 ml of the extract into a reaction tube and centrifuge at high speed in a microcentrifuge for 10 min)
transfer the supernatant into a sealable tube
further dilute the sample 1:25 (40 μl+960 μl) with buffer, e.g. pH neutral phosphate buffer: the final dilution factor is 1000.

The samples thus obtained are then used for total gluten determination preferably in a sandwich ELISA. The implementation of a sandwich ELISA for determining total gluten of wheat-containing samples is described below by way of example.

An essential advantage of the invention is that the total gluten content is determined with a single measurement step in a single cavity. For this purpose, the cavities of a commercially available microtiter plate are first coated with specific capture antibodies against gliadin (wheat prolamin), LMW and HMW glutenin (wheat lutelins); typically with concentrations between 0.5 and 5 μg/ml, each cavity being coated with all antibodies. Non-specific binding is saturated by block and stabilizing solutions known from literature and the extracted sample or standard (calibrator) is then added to the individual wells, preferably as duplicates. For the production of the calibrator, the gluten proteins are isolated from a wheat flour using modified Osborne fractionation (Schalk et al. 2017). To improve the shelf life, the gluten isolate obtained in this way is dried and stored. To prepare the calibrators, this isolate is weighed in, dissolved with cocktail extraction solution (EP 1 424 345 A1) R-Biopharm (product number R7006) and diluted to the desired concentrations in a pH-neutral phosphate buffer.

After sample or calibrator addition and a subsequent washing step with a common washing buffer (e.g. PBS/Tween), detection antibodies against gliadin (wheat prolamine), LMW- and HMW-glutenin (wheat glutenin) are added, each conjugated to a reporter enzyme. This mixture of detection antibodies, which bind to different sites on the gluten proteins than the capture antibodies, is hereinafter referred to as a combination conjugate. (A detailed description of the preparation of the combination conjugate according to the invention is given in the section “Preparation of the antibody combination conjugate”). After subsequent incubation, the individual wells are emptied, washed, filled with substrate reagent and, for the quantification of the gluten content, i.e. the quantitative determination of the proportion of gluten proteins bound to the detection antibodies, by measuring the substrate turnover by the reporter enzymes coupled to the detection antibodies.

In one embodiment, determining the concentration of the gluten proteins is carried out with a sandwich ELISA using a microtiter plate with a desired number of wells, wherein each individual well of the microtiter plate is coated with a mixture of the specific capture antibodies preferably against prolamins and against glutelins. The extracted food sample or a calibrator is added to a respective well and incubated. After incubation, the wells are washed to remove unbound sample material and then a uniform combination conjugate of conjugated detection antibodies against prolamine and gluteline are added to the wells. The total amount of gluten proteins in a single well is then determined by measuring the signal strength of the conjugated detection antibodies in comparison to the signal strengths of the calibrators carried along in the microtiter plate.

The examples of a sandwich ELISA as described in more detail above are possible procedures. Other possible methods include competitive ELISA (antigen coating or antibody coating), direct ELISA, as well as lateral flow, flow-through, microarray and microfluidic formats. These procedures are briefly described below.

In a competitive ELISA with antigen coating, the microtitre plate is coated with a gluten extract, e.g. from a wheat flour. The calibrator is identical to that used in the sandwich ELISA method. The preparation as well as the adjustment of the single conjugates and the combination conjugate are carried out in the same way as in the sandwich ELISA method, but the OD decreases with increasing gluten concentrations in a competitive ELISA. The sample extraction is identical to that of the sandwich ELISA method.

In the ELISA test procedure, calibrators or samples are placed in the microtiter plate wells in the first step and the combination conjugate is then added directly and calibrator or samples and conjugate are incubated together. However, the horseradish peroxidase is not stable to the cocktail extraction solution in the samples. Therefore, in this case, the antibodies have to be conjugated differently, for example to biotin. After a further washing step, the detection is carried out by means of horseradish peroxidase Streptavidin. Alternatively, non-conjugated antibodies are used and, after the washing step, the detection is carried out by means of horseradish peroxidase coupled secondary antibodies. In both cases, the colour reagent is added after a renewed washing step. In the last step, the reaction is terminated by adding the stop solution.

In a competitive ELISA with antibody coating, the microtiter plate is coated with a solution of prolamin, LMW and HMW antibodies, so that all antibodies are bound in each cavity. The calibrator is identical to that of the sandwich ELISA method. The individual protein fractions prolamine, LMW and HMW are coupled to horseradish peroxidase as conjugates. The individual conjugates and the combination conjugate are adjusted analogously to the sandwich ELISA method, but the OD decreases with increasing gluten concentrations in a competitive ELISA. The sample extraction is identical to that in the sandwich ELISA method.

In the ELISA test procedure, calibrators or samples are placed in the cavities and incubated in the first step. After an optional washing step, the combination conjugate is pipetted into the cavities and incubated. After a (renewed) washing step, the colour reagent is added and incubated. In the last step, the reaction is terminated by adding the stop solution.

Alternatively, a joint incubation of calibrators or samples with the combination conjugate can also be carried out. However, the horseradish peroxidase is not stable to the cocktail extraction solution in the samples. Therefore, in this case, the prolamin, LMW and HMW fraction must be conjugated differently, for example to biotin. After a further washing step, the detection is carried out by means of streptavidin coupled to horseradish peroxidase. After a renewed washing step, the colour reagent is added and incubated. In the last step, the reaction is terminated by adding the stop solution.

In a direct ELISA, an uncoated microtiter plate is used. The calibrator is identical to that of the sandwich ELISA method. The production and adjustment of the individual conjugates and of the combination conjugate are carried out as in the sandwich ELISA method. The sample extraction is identical to that in the sandwich ELISA method.

In the first step of the ELISA test procedure, the uncoated microtiter plate is incubated with the calibrators or samples. After a washing step, the combination conjugate is next added and incubated.

After a renewed washing step, the colour reagent is added and incubated. In the last step, the reaction is terminated by adding the stop solution.

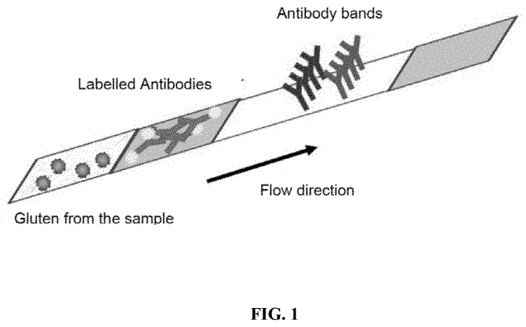

Lateral-flow formats can follow the sandwich or competitive principle. The former involves coating the surface of a strip-shaped cut membrane with a solution of a mixture of prolamin, LMW and HMW antibodies in the form of a narrow line transverse to the direction of travel (see FIG. 1). The invention also relates to a device for carrying out the inventive method. The gluten-containing sample solution is transported via an absorbent material into the region of the lateral flow in which a mixture of marked prolamin, LMW and HMW antibodies are located in dried form. The conjugated detection antibodies used against prolamines and glutelins in the combination conjugate are mixed according to the invention in such a way that all gluten fractions are measured with the same mass fraction with comparable signal strength. The marking of the antibodies follows the experts of known methods (eg in the form of colloidal gold, coloured, fluorescent or phosphorescent nanoparticles). The gluten proteins react with the respective labelled specific antibody and are transported further to the region of the membrane by capillary forces which has been coated with membrane-bound prolamin, LMW and HMW antibodies as described above. There, the gluten-antibody complexes react with the membrane-bound antibodies and thus form a sandwich which becomes visible through the formation of a coloured band. The signal strength of the band corresponds to the amount of gluten in the sample. Another band serves as a control band and indicates the general functionality of the lateral flow.

The competitive lateral flow format follows the principles of the competitive ELISA already described, wherein the gluten from the sample reacts first with marked specific antibodies and is then transported along the membrane by capillary forces until it meets a sample band with immobile gluten. A reduction in the intensity of the sample band indicates the presence of gluten. The labelled antibodies used against prolamines and glutelins in the combination conjugate are mixed according to the invention in such a way that all gluten fractions are measured with the same mass fraction with comparable signal strength. Alternatively, there is marked gluten on the starting line of the lateral flow, which together with the unlabelled gluten from the sample, transported by capillary forces, meets the test band with immobilised specific unlabelled antibodies. Here a competitive reaction for the antibody binding sites takes place. The mixed antibodies used against prolamines and glutelines in the test band and the labelled mixed gluten proteins on the start line of the lateral flow are mixed in accordance with the invention in such a way that all gluten fractions are measured at the same mass fraction with comparable signal strength. As a result, the band colours more intensively when there is less gluten in the sample.

In principle, flow-through formats follow the functional principles of lateral flow formats already described, differ only in the fact that the material transport does not take place along a membrane but through it (“through”). Adaptations to the lateral flow rates described can be taken from the relevant literature or are known to the expert.

Microarray and microfluidic formats follow the principles of ELISA and lateral flow formats already described. Further specific details can be gathered from the relevant literature or are known to the expert.

Preparation of the Antibody Combination Conjugate:

For the exemplary embodiment of the determination of the total gluten content from wheat-containing samples, a gliadin antibody is used as prolamin detection antibody against the epitope QQPFP and an antibody against the LMW fraction extracted from wheat flour and an antibody against HMW with the epitope recognition sequence GYYPTS is used as glutelin detection antibody.

The various detection antibodies are separately coupled to horseradish peroxidase using commercial conjugation reagents (eg Pierce™ Conjugate Purification Kit catalogue number 44920). The antibody conjugates thus obtained are then mixed according to the invention in such a way that all gluten fractions are measured with the same mass fraction with comparable signal strength. For this purpose, the combination conjugate with dilution series of Prolamin, LMW and HMW fractions from wheat were tested. If no comparable signal strength is achieved, the mixing ratio of the individual conjugates is adapted according to their reactivities to the wheat fractions in the combination conjugate. For this purpose, it may be helpful to first prepare the individual conjugates separately from one another and to estimate the dilution factors of the individual conjugates in a preliminary test (see Table 1). The individual conjugates are then mixed in the small batch and the reactivity of the combination conjugate is checked as described above and the dilution factors of the individual conjugates are optionally further adjusted. Only after successful adjustment in the small batch are the individual conjugates combined as a whole.

Table 1 shows the example of such a testing. For this purpose, the specific antibody conjugates are first tested individually with the above mentioned coated microtiter plate. A total wheat gluten extract is diluted to three different concentrations, each containing the same amount of either prolamin, LMW or HMW proteins. Thus, a total gluten extract with a concentration of 15.5 ng/ml total gluten protein contains the equivalent of 10 ng/ml gliadin. Accordingly, a total gluten extract with the concentration 44.5 ng/ml total gluten protein contains 10 ng/ml LMW proteins and a total gluten extract with the concentration 80 ng/ml contains 10 ng/ml HMW proteins (see Table 1).

TABLE 1
Selection of total gluten concentrations in which
the concentration of the respective individual
fractions has the same value. All data in ng/ml.

| | Prolamine | LMW | HMW |
|---|---|---|---|
| Total | 65% | 22.50% | 12.50% |
| Wheat gluten | of total gluten | of total gluten | of total gluten |
| 0 | 0.0 | 0.0 | 0.0 |
| 1.3 | 0.8 | 0.3 | 0.2 |
| 2.5 | 1.6 | 0.6 | 0.3 |
| 5.0 | 3.3 | 1.1 | 0.6 |
| 10.0 | 6.5 | 2.3 | 1.3 |
| 15.5 | 10.1 | 3.5 | 1.9 |
| 20.0 | 13.0 | 4.5 | 2.5 |
| 40.0 | 26.0 | 9.0 | 5.0 |
| 44.5 | 28.9 | 10.0 | 5.6 |
| 80.0 | 52.0 | 18.0 | 10.0 |

The gliadin, LMW and HMW-specific antibody conjugates are now adjusted in such a way that the gliadin antibody with the 15.5 ng/ml total gluten extract has the same signal strength (in the example in Table 2: 2.0) such as the LMW antibody with the 44.5 ng/ml total gluten extract and the HMW antibody with the 80 ng/ml total gluten extract (Table 2).

TABLE 2
Example of the preliminary testing of single conjugate dilutions.

| | Prolamine | LMW | HMW |
|---|---|---|---|
| Total gluten concentration (ng/ml) | 15.5 | 44.5 | 80.0 |
| Concentration Target protein | 10.1 | 10.0 | 10.0 |
| fraction (ng/ml) | | | |
| Single conjugate to be tested | Prolamine | LMW | HMW |
| OD at dilution 1 of each individual | 3.0 | 2.4 | 4.0 |
| Conjugate | | | |
| OD at dilution 2 of each individual | 2.5 | 2.0 | 3.33 |
| Conjugate | | | |
| OD at dilution 3 of each individual | 2.0 | 1.6 | 2.66 |
| Conjugate | | | |
| OD at dilution 4 of each individual | 1.5 | 1.2 | 2.0 |
| Conjugate | | | |
| OD at dilution 5 of each individual | 1.0 | 0.8 | 1.33 |
| Conjugate | | | |

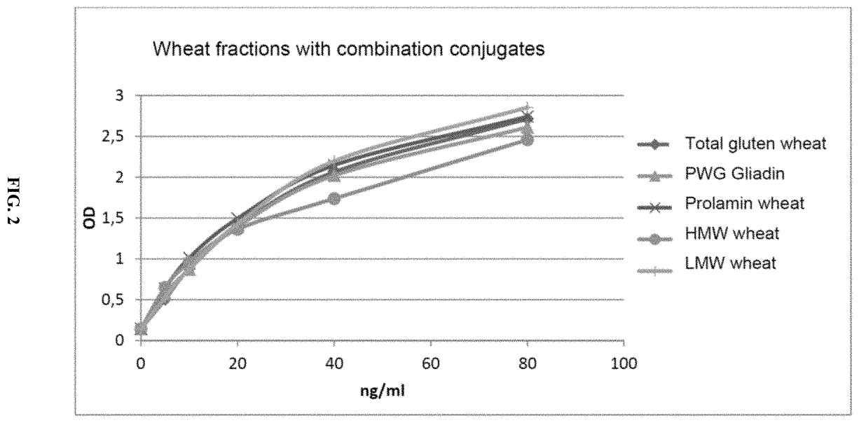

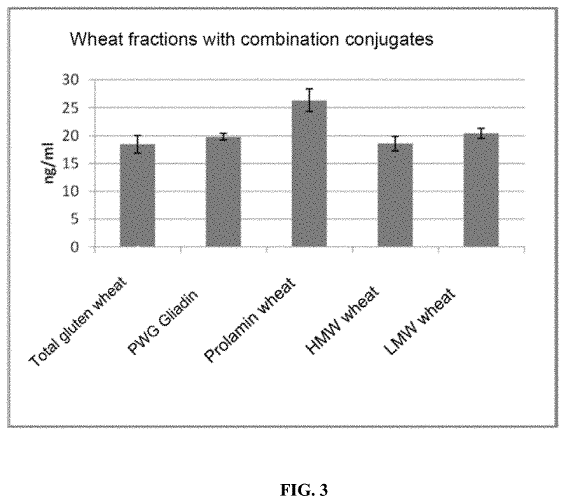

The combination conjugate is then checked with calibration series of the individual fractions (wheat prolamine, LMW and HMW proteins) (see FIG. 2 and FIG. 3). Optionally, a further adaptation of the composition of the combination conjugate is carried out. The individual fractions are obtained by extraction of all proteins from a wheat flour and subsequent separation in HPLC (Schalk et al. 2017) to improve the shelf life, the protein isolates obtained in this way are dried and stored. To prepare the stock solutions, the fractions are weighed, dissolved by means of cocktail extraction solution (EP 1 424 345 A1) and diluted to the desired concentrations in a pH-neutral phosphate buffer.

A detailed exemplary embodiment of a sandwich ELISAs for quantitative gluten determination from a cereal or food sample Z is also quoted in the following:
Each 100 μl of the standard solution or the prepared samples are pipetted into the corresponding cavities in a double determination and incubated for 20 minutes at room temperature (20-25° C.).
Empty the cavities by tapping out the liquid and remove the residual liquid by forcefully tapping out (three times in succession) on absorbent laboratory cloths. The cavities each wash with 250 μl washing buffer (PBS/Tween). Repeat this process twice.
pipette each 100 μl of the combination conjugate into the corresponding cavities and Incubate for a further 20 minutes at room temperature (20° C.).
Empty the cavities by tapping out the liquid and remove the residual liquid by forcefully tapping out (three times in succession) on absorbent laboratory cloths. The cavities each wash with 250 μl of wash buffer. Repeat this process twice.
Each 100 μl of a commercially available horseradish peroxidase substrate/chromogen solution is pipetted into the cavities. Mix carefully and incubate for 10 minutes at room temperature (20-25° C.) in the dark.
Pipette each case 100 μl of stop solution into each cavity and carefully mixed carefully. The absorbance at 450 nm within 30 min after addition of the stop solution is measured in a commercially available photometer.

Quantification of the Total Gluten Content of Various Wheat Flour:

Wheat flours can differ significantly both in the total protein content and in the composition of the gluten proteins (Hajas et al. 2017). Due to the adjustment of the individual conjugates, differences in the composition of the gluten proteins should have no influence on the measurement, since the same amount of individual fraction protein leads to a comparable signal, regardless of which wheat protein fraction it is. In contrast to conventional test methods (see prior art), the total gluten content in the case of different gluten protein compositions is not falsified by determining all the celiac disease-relevant gluten protein fractions.

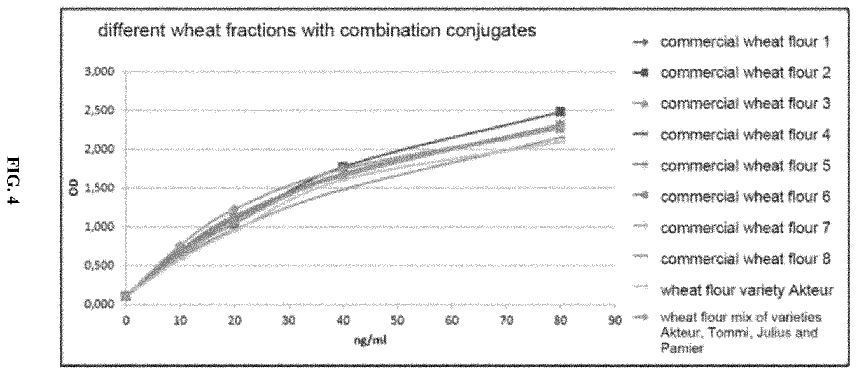

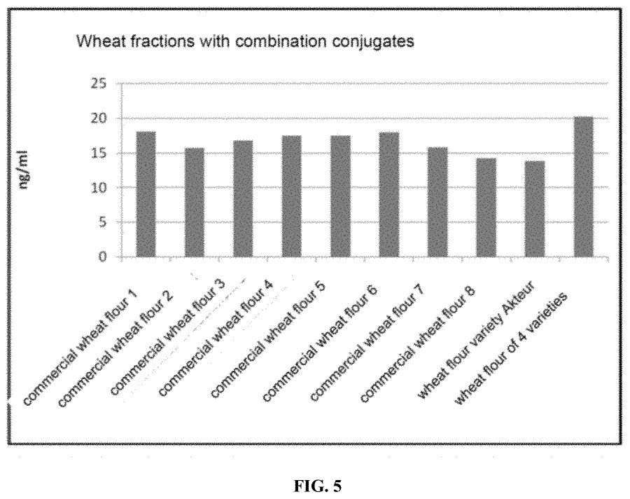

Nine wheat flours and one wheat flour mix were extracted with the cocktail extraction solution from EP 1 424 345 A1 for the determination of the total gluten content of various wheat meals. The extracts were diluted in the extraction medium and a pH neutral sample dilution buffer to the concentrations 0; 5; 10; 20; 40 and 80 ng/ml total gluten protein. The gluten protein contents of the different flours had previously been determined by HPLC (Schalk et al. 2017). FIG. 4 and FIG. 5 show that all wheat flours are found within acceptable ranges, especially when the extremely high dilution factor required for the measurement of the extracts is taken into account.

Quantitative determination of the total gluten content in other cereals.

Other cereal flours can also differ significantly both in their total protein content and in the composition of the gluten proteins. The overall reactivity of the measuring system depends on the strength of the individual cross-reactivities of the antibodies used to other grains. For example, the prolamines from wheat, rye and barley show very large sequence homologies, so that a high level of cross-reactivity of the prolamine antibody can be expected here. Furthermore, the HMW proteins from wheat and rye show large sequence homologies, so that a high level of cross-reactivity of the wheat HMW antibody can be expected here as well. Depending on which sequences exactly the prolamine and HMW antibodies recognise and how frequently these sequences occur in the cereals, a reactivity of the antibodies which exceeds the reactivity towards wheat can also be expected. LMW proteins have no homologous proteins in rye and barley, so that cross-reactivity is not to be expected here. Barley glutinins have hardly any homologies to wheat and rye proteins, so that detection of this fraction with antibodies against prolamins, HMW and LMW proteins is not likely.

Various commercial flours and pure varieties with the cocktail extraction solution from EP 1 424 345 A1 are extracted for checking the reactivity with respect to rye and barley meals. The extracts are diluted in extraction medium and a pH neutral sample dilution buffer to concentrations 0; 5; 10; 20; 40 and 80 ng/ml total gluten protein. The gluten protein contents of the various flours were previously determined by means of HPLC (Schalk et al. 2017).

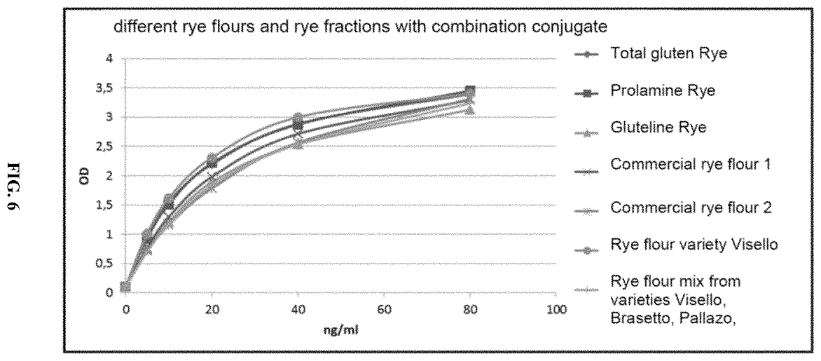

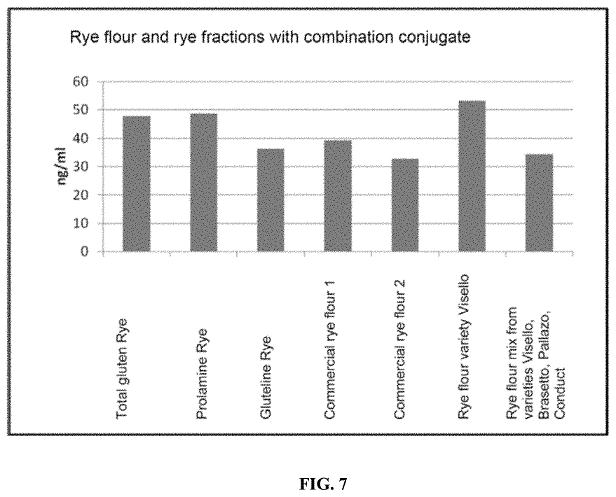

FIG. 6 shows that all rye flours are detected relatively similarly. When calculating the measured concentrations, it is found that all rye flours are overdetermined (FIG. 7). This is based on the increased reactivity of the prolamina antibody used in relation to rye prolaminae.

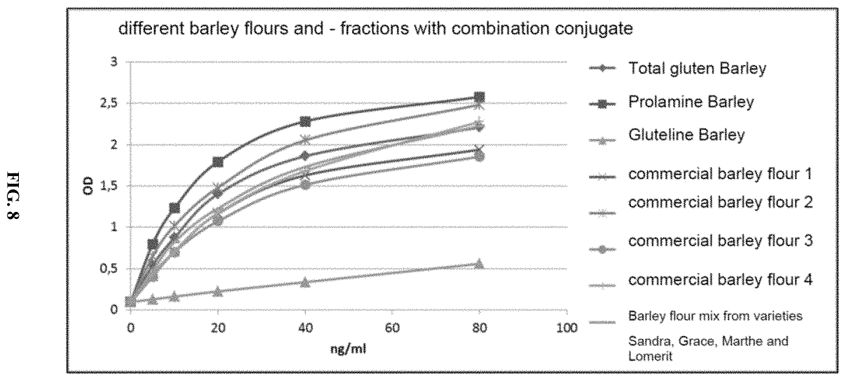

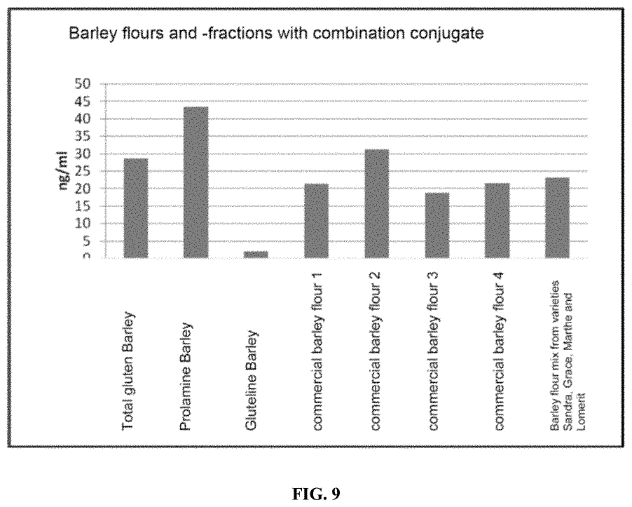

In contrast to wheat and rye flours, barley flours show a greater range of variation (FIG. 8 and FIG. 9). This is probably due to the fact that the barley fractions are detected differently from the wheat and rye fractions. As was to be expected, the barley gluten fraction is not significantly detected by any of the antibodies used, while the prolamines are detected very well. Accordingly, slight shifts in the protein fractions would lead to significant differences in the various flours.

In case of a general over- or underdetermination of cereals compared to a wheat calibrator material, a calibrator with material from the corresponding other cereal can be used for a more precise gluten content determination.

By using the combination conjugate according to the invention, the claimed method allows the simultaneous determination of prolamine and glutelin proteins and thus allows for the first time a quantification of the actual total gluten content. This method of a joint determination of prolamine and glutelin proteins is much more accurate than the methods known up to now, in which only the prolamine content is determined, from which in turn the total gluten content is then calculated. The glutelines, which are also relevant for celiac disease, have not been determined so far, which in the past could lead to incorrect results. The claimed method closes this gap.

Furthermore, the method according to the invention is not only suitable for the determination of the total gluten content of food samples containing wheat, but also for the determination of the total gluten content of other food samples containing Triticum species and/or rye and/or barley.

The present invention also relates to a test kit for determining the total gluten content of food samples. The test kit for the quantification of total gluten proteins from wheat and/or Triticum species and/or barley and/or rye in food samples may comprise a microtiter plate coated with capture antibodies, a wheat flour extract as a calibrator, a combination conjugate with horseradish peroxidase-conjugated detection antibodies against the above-mentioned gluten protein proteins fractions from wheat, rye and barley, wherein the combination conjugate is diluted in such a way that all gluten fractions proteins are measured at the same mass fraction with comparable signal strength, a colour reagent (substrate), a stop solution, a sample dilution buffer and a wash buffer.

### CITED DOCUMENTS

Anonymous (2014) of ALL-TEK™ Gluten ELISA Assay Instructions for use, catalogue number SE110019, manufactured by ELISA Technologies, Inc, 2501 NW 66th Court, Gainesville, Fla. 32653 USA.
Hajas, L, Scherf, K, Török, K, Bugyi, Z, Sound, E, Poms, R, Koehler, P and Killing Közi, p. (2018) Variation in protein composition among wheat (Triticum aestivum L) cultivars to identify cultivars suitable as reference material for wheat gluten analysis. Food Chem 267: 387-3394.
Lexhaller, B, Tompos, C and Scherf, K A (2016) Comparative analysis of prolamin and glutelin fractions from wheat, rye, and barley with five sandwich ELISA test kits. Anal Bioanal. Chem. 498: 6093-6104.
Schalk, K, Lexhaller, B, Koehler, P and Scherf, K (2017) Isolation and characterization of gluten protein types from wheat, rye, barley and oats for use as reference materials. PLOTTING ONE 12 (2): E0172819.
Skerritt, J H, Jenkins, K L and Hill, A S (1989) Monoclonal antibody Based Two-site Enzymes Immunoassays for Wheat Gluten Proteins. 2. Specificity Analysis. Food Agric. Immunol. 1: 161-171.
Tye-Din, J, Stewart, J, Dromey, J, Beissbarth, T, van Heel, D, Tatham, A, Henderson, K, Mannering, S, Gianfrani, C, Jewell, D, Hill, A, McCluskey, J, Rossjohn, J and Anderson, R (2010) Comprehensive, Quantitative Mapping of T Cell Epitopes in Gluten in Celiac Disease. Let. Trans. Med. 2: 41 RA51.
Wieser, H and Koehler, P (2009) Is the calculation of the gluten content by multiplying the prolamin content by factor 2 valid? Eur. Food Res Technol. 229: 9-13.
Wieser, H, Koehler, P and Konitzer, K (2014) Celiac Disease and Gluten. Academic Press, ISBN 978-0-12-420220-7.

1. A method for the quantification of total gluten proteins from cereals in food samples comprising,
extracting gluten proteins from the food samples; and determining the total concentration of the gluten proteins with a combination of specific affinity reagents, wherein the combination of specific affinity reagents comprise a combination of detection antibodies against prolamine and gluteline mixed together in one unit, and wherein determining the concentration of the gluten proteins occurs in a single analytical run.

2. The method according to claim 1, wherein determining the concentration of the gluten proteins is carried out with an ELISA, an immunoturbidimetric assay, a lateral flow assay, a flow-through assay, a microarray assay or a microfluidic assay, using the combination of antibodies against prolamine and gluteline.

3. The method according to claim 1, wherein the specific affinity reagents further comprise antibody fragments, nanobodies, aptamers, specific receptors, T-cell receptors and/or Molecularly Imprinted Polymers (MIP).

4. The method according to claim 1, wherein determining the concentration of the total gluten proteins includes using a sandwich ELISA with a microtiter plate possessing a desired number of wells, wherein each well of the microtiter plate is coated with a mixture of specific capture antibodies against prolamins and glutelins, wherein the food sample or a calibrator is added to a respective well, wherein unbound sample material is removed after incubation, then the combination of specific affinity reagents comprising the detection antibodies against prolamine and gluteline is added to the respective well, and wherein the total amount of gluten proteins in a single well is determined by measuring the signal strength of the detection antibodies in comparison to the signal strengths of the calibrators.

5. The method according to claim 4, wherein the detection antibodies used against prolamine and gluteline are mixed in the combination in such a way that all gluten fractions (gliadins, LMW-glutenin subunits and HMW-glutenin subunits each from wheat, prolamine from rye and barley and HMW-secaline from rye) are measured at the same mass fraction with comparable signal strength.

6. The method according to claim 1, wherein determining the concentration of the total gluten proteins further comprises using a calibrator and wherein the gluten proteins contain at least one member of the group consisting of wheat, rye, barley, a Triticum grain, and combinations thereof.

7. A method according to claim 1, wherein of the gluten proteins in the food samples contain wheat and/or other Triticum species and/or rye and/or barley.

8. A test kit for the quantification of total gluten proteins from wheat and/or Triticum species and/or barley and/or rye in food samples comprising a microtiter plate coated with capture antibodies, a wheat flour extract as a calibrator, a combination conjugate with horseradish peroxidase-conjugated detection antibodies against the gluten proteins from wheat, rye and barley, wherein the combination conjugate is diluted in such a way that all gluten proteins are measured at the same mass fraction with comparable signal strength, a colour reagent (substrate), a stop solution, a sample dilution buffer and a wash buffer.
